(19) United States
(12) Patent Application Publication
(10) Pub. No.: US 2010/0041615 A1
(43) Pub. Date: Feb. 18, 2010

(54) PROTEIN-BINDING METHOTREXATE DERIVATIVES, AND MEDICAMENTS CONTAINING THE SAME

(75) Inventors: Felix Kratz, Ihringen (DE); Andre Warnecke, Freiburg (DE)

Correspondence Address: KNOBBE MARTENS OLSON & BEAR LLP, 2040 MAIN STREET, FOURTEENTH FLOOR, IRVINE, CA, 92614, US

(73) Assignee: Medac Gesellschaft Fur Klinische Spezialpraparate MBH

(21) Appl. No.: 12/375,406

(22) PCT Filed: Jul. 25, 2007
(86) PCT No.: PCT/EP2007/006618

§ 371 Date: Sep. 29, 2009

(30) Foreign Application Priority Data
Jul. 28, 2006 (DE) 10 2006 035 083.9

(51) Int. Cl. A61K 38/07; C07K 5/10

(52) U.S. Cl. 514/18; 530/330

(57) ABSTRACT

The invention relates to methotrexate derivatives which contain a protein-binding group and can be enzymatically cleaved in the body such that the active substance or a low-molecular active substance derivative is released. Also disclosed is a method for producing methotrexate derivatives, the use thereof, and medicaments comprising methotrexate derivative.

The present invention relates to methotrexate derivatives and methotrexate peptide derivatives, which contain a protein-binding group and can be enzymatically cleaved in the body such that the active substance or a low-molecular active substance derivative is released, a method for producing methotrexate derivatives, their use, and medicaments containing methotrexate derivatives.

Methotrexate (MTX) is a folic acid antagonist used in the treatment of tumors and rheumatoid arthritis. Its use is limited by a number of side effects (e.g. vertigo, alopecia, stomatitis, gastrointestinal symptoms, increased infection susceptibility). In order to improve the side effect profile and the effectiveness of MTX and MTX derivatives, macromolecular transport forms of MTX have been provided by coupling the active substance to synthetic polymers, such as poly(ethylene glycol) (Riebeseel, K.; Biedermann, E.; Löser, R.; Breiter, N.; Hanselmann, R. et al., Bioconjugate Chem. 2002, 13, 773-785), HPMA copolymers (Subr, V.; Strohalm, J. et al. Controlled Release 1997, 49, 123-132) or human serum albumin (HSA) (Wunder, A.; Muller-Ladner et al., J Immunol 2003, 170, 4793-4801; Wunder, A.; Stehle, G. et al., Int. J. Oncol. 1997, 11, 497-507). However, there is still a demand for new systems containing MTX or MTX derivatives, which have a low side effect profile and an essentially improved effectiveness compared to free MTX.

Thus, the technical problem underlying the present invention is to provide prodrugs of methotrexate releasing MTX or MTX derivatives in tumorous tissue or rheumatoid tissue.

This technical problem is solved by the embodiments characterized in the claims.

In particular, methotrexate derivatives of the general structural formula

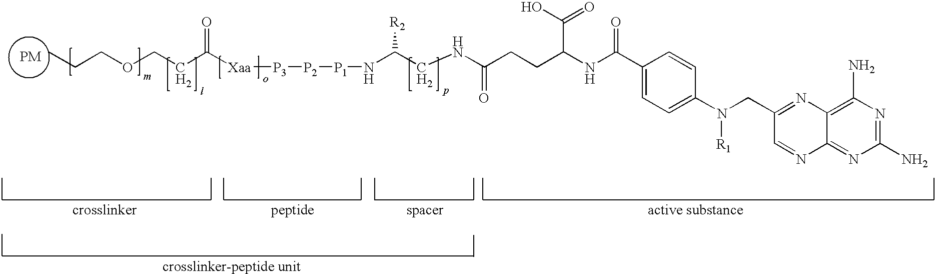

are provided, wherein R₁=H or CH₃, R₂=H or COOH, P₁-P₃=L- or D-amino acids, Xₐₐ is a solubility-mediating amino acid, m=0 to 6, n=0 to 5, o=0 to 2, p=1 to 10, and PM is a protein-binding group.

According to the present invention, an integrated hydrolytically or enzymatically cleavable, predetermined breaking point allows to release the active substance or a spacer-active substance derivative in vivo in controlled fashion, so that methotrexate derivatives of the present invention constitute prodrugs.

The MTX derivatives of the present invention are composed of an antitumor or antirheumatic methotrexate component, a spacer molecule, a peptide chain and a heterobifunctional crosslinker. This structural set-up will be explained in detail in the following:

The antitumor MTX component of the present invention is an active substance with the general structural formula

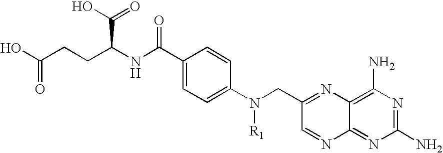

wherein

### R₁=CH₃ or H.

The preferred active substance is methotrexate.

The spacer molecule of the present invention is a diamine with the general structural formula

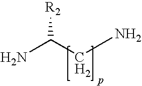

wherein

### R₂=H or COOH

p=1 to 10.

Preferred spacers are ethylenediamine (R₂=H, p=1) and spacer in which p=4 or 5. A particularly preferred spacer is L-lysine (R₂=COOH, p=4).

In the present invention, the peptide is composed of an enzymatically cleavable sequence and an N-terminal solubility-mediating component, and has the general structural formula

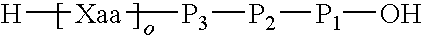

wherein
P₁-P₃=L- or D-amino acids
Xₐₐ=an amino acid with an alkaline side chain
o=0-2.

In the present invention, the amino acid P₁ is selected from the amino acids lysine, methionine, alanine, proline and glycine. The amino acid P₂ is selected from the amino acids leucine, phenylalanine, methionine, alanine, proline and tyrosine. The amino acid P₃ is selected from the amino acids D-alanine, alanine, D-valine, valine, leucine and phenylalanine. Preferred amino acids in the P₁ position are lysine, alanine and methionine. Preferred amino acids in the P₂ position are phenylalanine, methionine, alanine and tyrosine. Preferred amino acids in the P₃ position are D-alanine, alanine, D-valine, valine and phenylalanine.

Particularly preferred peptide sequences are listed in the table below.

| P₃ | P₂ | P₁ |
|---|---|---|
| D-Ala | Phe | Lys |
| Ala | Phe | Lys |
| D-Val | Leu | Lys |
| Val | Leu | Lys |
| Ala | Phe | Met |
| Phe | Ala | Met |
| Ala | Met | Met |
| Phe | Met | Met |

According to the present invention, the solubility-mediating group Xaa is preferably selected from the amino acids arginine, lysine and histidine. A particularly preferred group is arginine.

In the present invention, the heterobifunctional crosslinker is a carboxylic acid having a protein-binding group with the general structural formula

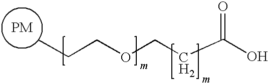

wherein
m=0 to 6
n=0 to 5
PM=protein-binding group.

The protein-binding group (PM) is preferably selected from a 2-dithiopyridyl group, a halogen acetamide group, a halogen acetate group, a disulphide group, an acrylic acid ester group, a monoalkyl maleic acid ester group, a monoalkyl maleamine acid amide group, an N-hydroxy succinimidyl ester group, an isothiocyanate group, an aziridine group or a maleinimide group. A particularly preferred protein-binding group is the maleinimide group.

Preferred crosslinkers are characterized by m=3 and n=1 as well as by m=0 and n=4.

According to the present invention, the active substance and the spacer molecule are linked by an amide bond between the γ-carboxyl group of the active substance and the first amino group of the spacer molecule. The bond between the spacer molecule and the crosslinker-peptide unit consists of an amide bond between the second amino group of the spacer molecule and the C-terminal carboxyl group of the crosslinker-peptide unit. The bond between the crosslinker and the peptide chain consists of an amide bond between the N-terminus of the peptide chain and the carboxyl group of the crosslinker.

An essential property of the MTX derivatives of the present invention is that the bond between the spacer molecule and the crosslinker can be cleaved enzymatically, whereby a controlled release of the active substance or a spacer-active substance derivative in tumorous tissue or rheumatoid tissue is allowed. Proteases, such as cathepsins or plasmin, are overexpressed in many human tumors and rheumatoid tissue, thus representing an ideal point of application for a target-oriented, enzymatic activation of prodrugs (Yan, S. et al., Biol. Chem. 1998, 2, 113-123; Leto, G. et al., Clin. Exp. Metastasis 2004, 91-106; Sloane, B. F.; Yan, S. et al., Seminars in Cancer Biology, 2005, 15, 149-157; Dano, K.; Behrendt, N. et al., Thrombosis & Haemostasis 2005, 93, 676-681; Hashimoto, Y.; Kakegawa, H. et al., Biochem. Biophys. Res. Commun. 2001, 283, 334-339; Ikeda, Y.; Ikata, T. et al., J. Med. Invest. 2000, 47, 61-75). Moreover, the MTX derivatives of the present invention show a fast cleavage in experimental tumor homogenates and synovial fluids of patients suffering from rheumatoid arthritis (see examples 4 and 5).

The MTX derivatives of the present invention are preferably produced by condensation of methotrexate derivatives with the general structural formula

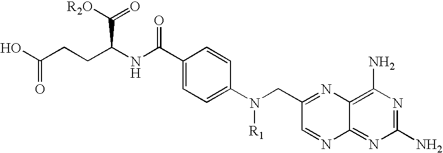

wherein

### R₁=CH₃, H or COCF₃

R₂=C(CH₃)₃, an alkoxy-substituted benzyl group or a trialkyl silyl group, with a crosslinker-peptide unit of the general structural formula

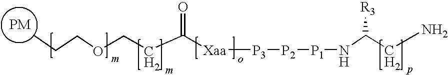

wherein

### R₃=H, COOH or COOtBu

P₁=lysine, methionine, alanine, proline or glycine
P₂=leucine, phenylalanine, methionine, alanine, proline or tyrosine
P₃=D-alanine, alanine, D-valine, valine, leucine or phenylalanine
Xₐₐ=amino acid with alkaline side chain
m=0 to 6
n=0 to 5
o=0 to 2
p=1 to 10
PM is a protein-binding group,
wherein possible nucleophilic groups are optionally present in protected fashion at P₁, P₂ and Xaa by protective groups known to the skilled person.

According to the present invention, as reagents for the activation of the carboxyl group of the crosslinker-peptide unit, preferably O-(azabenzotriazol-1-yl)-N,N,N′,N′-tetramethyluronium hexafluorophosphate (HATU), (benzotriazol-1-yloxy)tris(dimethylamino)phosphonium hexafluorophosphate (BOP), N,N′-diisopropyl carbodiimide (DIPC), N,N′-dicyclohexyl carbodiimide (DCC) or 2-chloro-1-methylpyridinium iodide are used with addition of common catalysts or auxiliary bases, such as N-ethyldiisopropylamine (DIEA), trialkylamine, pyridine, 4-dimethylaminopyridine (DMAP) or hydroxybenzotriazole (HOBt). The reaction is for example performed in a polar organic solvent, preferably in N,N-dimethyl formamide. The reactions are for example carried out at temperatures between −10° C. and room temperature, wherein the reaction time is e.g. between 30 min and 48 hours. Isolation of the intermediate product is for example achieved by precipitation from a non-polar solvent, preferably diethyl ether.

In a second subsequent synthesis step, according to the invention, the protective group R₂ together with possible protective groups for nucleophilic groups at P₁, P₂ and Xaa is removed. This cleavage is typically achieved by treatment with an acid, preferably trifluoroacetic acid or hydrogen chloride. In a preferred embodiment of the invention, the product of the first synthesis step is treated with a mixture of trifluoroacetic acid and dichloromethane in a ratio of 1:1 for about 30 min. The raw product is isolated by precipitation from a non-polar solvent, preferably diethyl ether.

According to the present invention, the raw product is purified e.g. by crystallization or column chromatography, preferably on reversed-phase silica gel.

According to a preferred embodiment of the present invention, methotrexate-γ-tert.-butylester is condensed with EMC-D-Ala-Phe-Lys(Boc)-Lys-OH (EMC=6-maleinimidocaproic acid) using HATU as a coupling reagent, and subsequently treated with trifluoroacetic acid (see example 1).

The protein-binding methotrexate derivatives of the present invention may be administered parenterally, preferably intravenously. To this end, the MTX derivatives of the present invention are provided as solutions, solids or lyophilisates, optionally using common pharmaceutically acceptable auxiliary agents, such as carriers, diluents or solvents. Examples of such auxiliary agents are polysorbates, glucose, lactose, mannitol, dextranes, citric acid, tromethamol, triethanolamine, aminoacetic acid or synthetic polymers or mixtures thereof. Preferrably, the MTX derivatives of the present invention are administered when dissolved in an isotonic buffer. The solubility of the MTX derivative may be optionally improved by means of pharmaceutically acceptable solvents, such as 1,2-propandiol, ethanol, isopropanol, glycerol or poly(ethylene glycol) having a molecular weight of 200 to 600 g/mol, or mixtures thereof, preferrably poly(ethylene glycol) having a molecular weight of 600 g/mol, or solubility mediator, such as Tween 80, Cremophor or polyvinylpyrrolidone, or mixtures thereof.

An essential property of the MTX derivatives of the present invention is the fast covalent bonding to serum proteins via a protein-binding group, whereby a macromolecular transport form of the active substance is generated. Serum proteins, such as transferrin, albumin and LDL, are known to have an increased take-up in tumorous tissue and accumulation in rheumatoid tissue (Kratz F., Beyer U., Drug Delivery 1998, 5, 281-299; Adams, B. K., Al Attia, H. M. et al., Nuclear Med. Commun. 2001, 22, 315-318; Sahin, M., Bernay, I. et al., Ann. Nuclear Med. 1999, 13, 389-395; Liberatore, M., Clemente, M. et al., J. Nuclear Med. 1992, 19, 853-857), so that they may be used as endogenous carriers for cytostatic agents within the scope of the present invention. A particularly preferred serum protein is circulating human serum albumin (HSA), which constitutes the major component of human blood with an average concentration of 30 to 50 g/L (Peters T., Adv. Protein Chem. 1985, 37, 161-245) and exhibits a free cysteine group (cysteine-34-group) on the surface of the protein, which is suitable for bonding thiol-binding groups, such as maleinimides or disulphides (WO 00/76551). The fact that maleinimide-functionalized MTX derivatives of the present invention bond fast and selectively to HSA is shown in Example 2. The reaction of the novel MTX derivatives with serum proteins may also be performed extracorporeally, e.g. with an albumin, blood or serum quantity provided for infusion.

In comparison to methotrexate conjugates having synthetic polymers as carrier systems, the MTX peptide derivatives of the present invention have the additional advantage that they are chemically unambiguously defined.

The figures show:

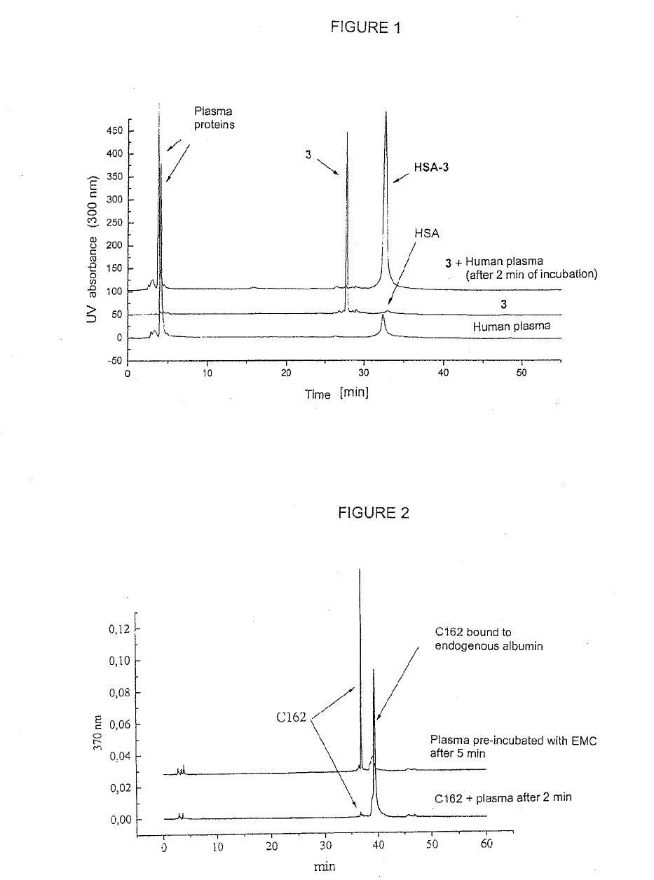

FIG. 1: chromatograms of human plasma, EMC-D-Ala-Phe-Lys-Lys(γ-MTX)-OH (3) and EMC-D-Ala-Phe-Lys-Lys(γ-MTX)-OH (3) after 2 min of incubation with human plasma at 37° C. (detection at λ=300 nm).

FIG. 2: chromatograms of EMC-Arg-Ala-Phe-Met-Lys(γ-MTX)-OH (C162) (200 μM) after 2 min of incubation with human plasma at 37° C. and after 5 min of incubation with human plasma having been preincubated with EMC (1000 μM) for 30 min.

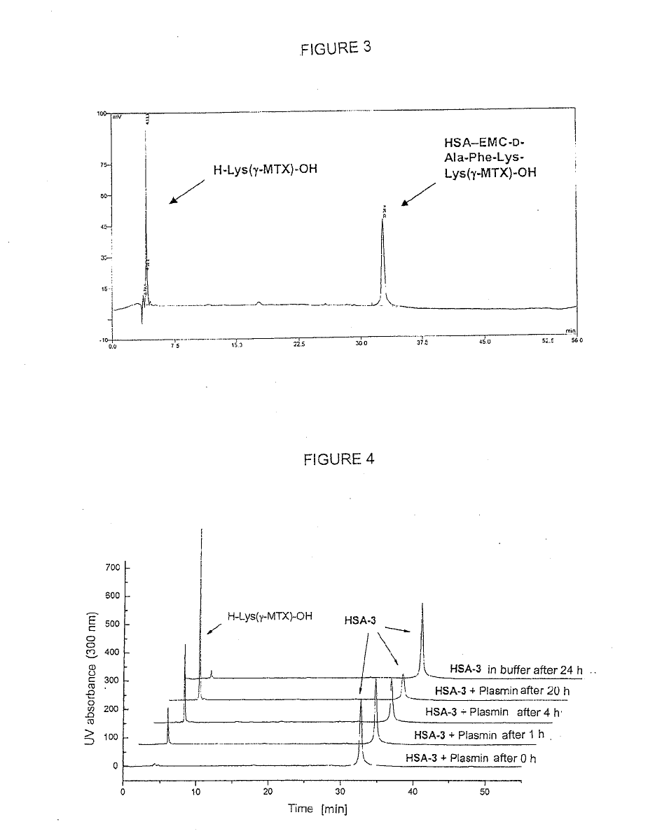

FIG. 3: a chromatogram of the HSA conjugate of EMC-D-Ala-Phe-Lys-Lys(γ-MTX)-OH after 4 hours of incubation with human plasmin (detection at λ=370 nm).

FIG. 4: chromatograms of the HSA conjugate of EMC-D-Ala-Phe-Lys-Lys(γ-MTX)-OH (3) after 0, 1, 4 and 20 hours of incubation with human plasmin and after 24 hours in buffer (detection at λ=300 nm).

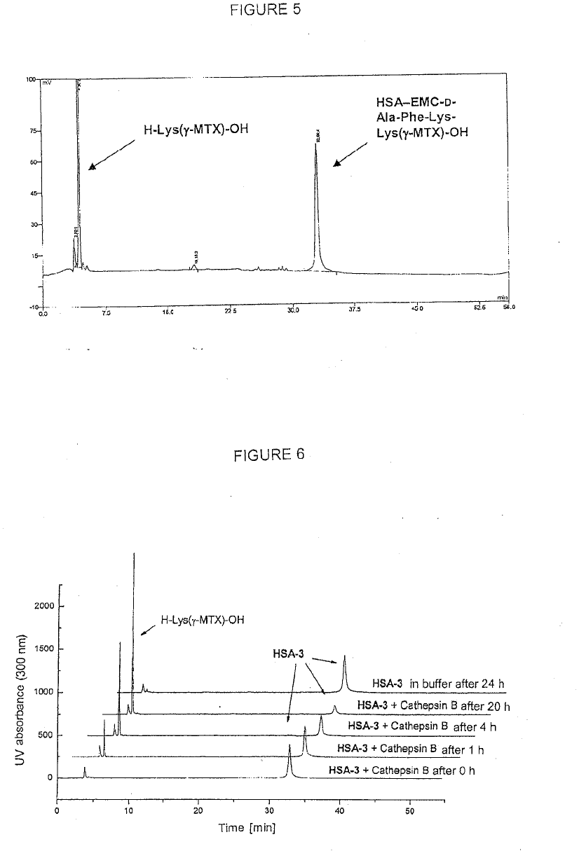

FIG. 5: a chromatogram of the HSA conjugate of EMC-D-Ala-Phe-Lys-Lys(γ-MTX)-OH after 4 hours of incubation with cathepsin B (detection at λ=370 nm).

FIG. 6: chromatograms of the HSA conjugate of EMC-D-Ala-Phe-Lys-Lys(γ-MTX)-OH (3) after 0, 1, 4 and 24 hours of incubation with cathepsin B and after 24 hours in buffer (detection at λ=300 nm).

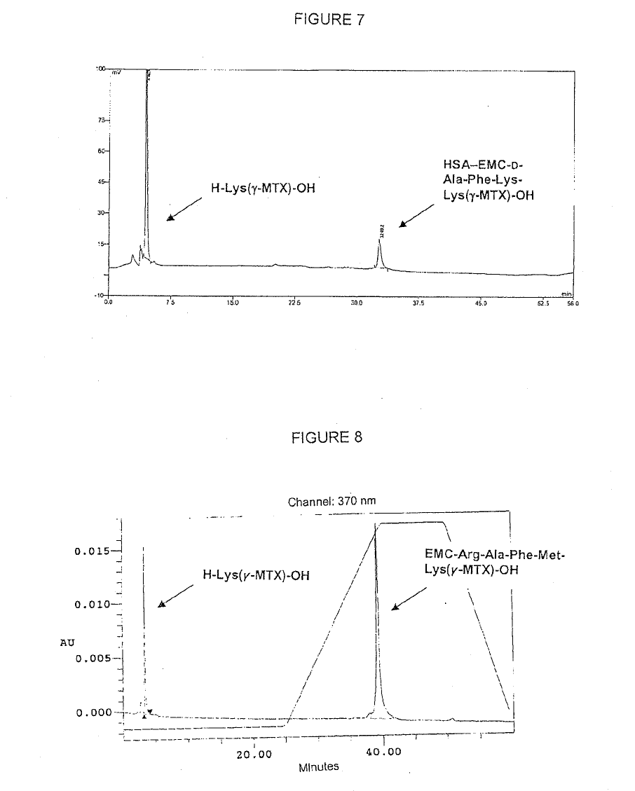

FIG. 7: a chromatogram of the HSA conjugate of EMC-D-Ala-Phe-Lys-Lys(γ-MTX)-OH after 4 hours of incubation with OVCAR-3 tumor homogenate (detection at λ=370 nm).

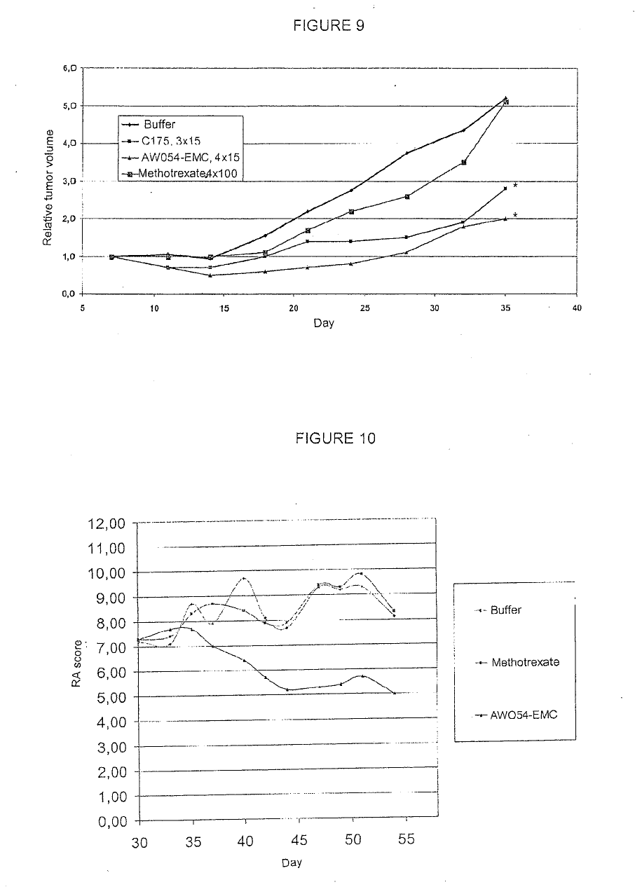

FIG. 8: a chromatogram of the HSA conjugate of EMC-Arg-Ala-Phe-Met-Lys(γ-MTX)-OH (C162) after 4 hours of incubation with synovial fluids of patients suffering from RA (detection at λ=370 nm).

FIG. 9: a graphical illustration showing the course of tumor growth in an OVCAR-3 model.

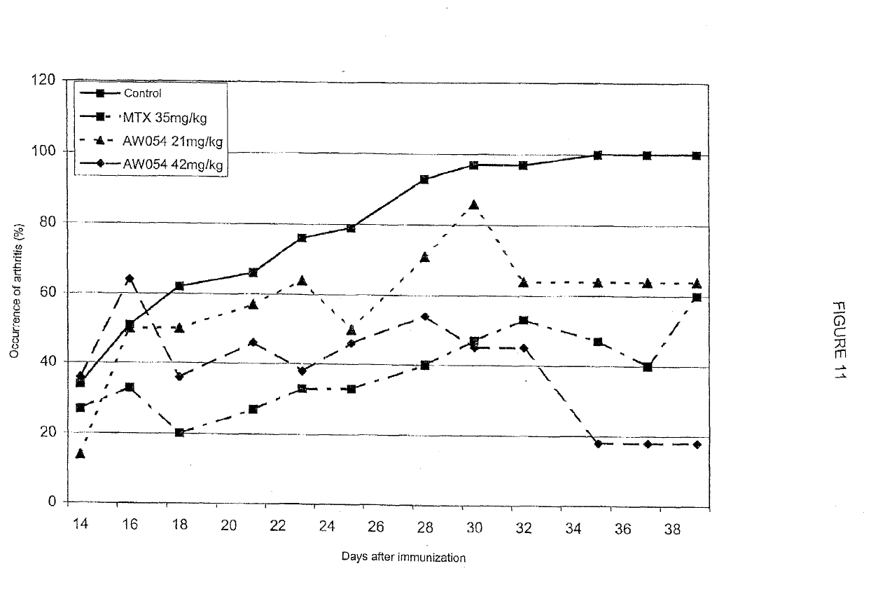

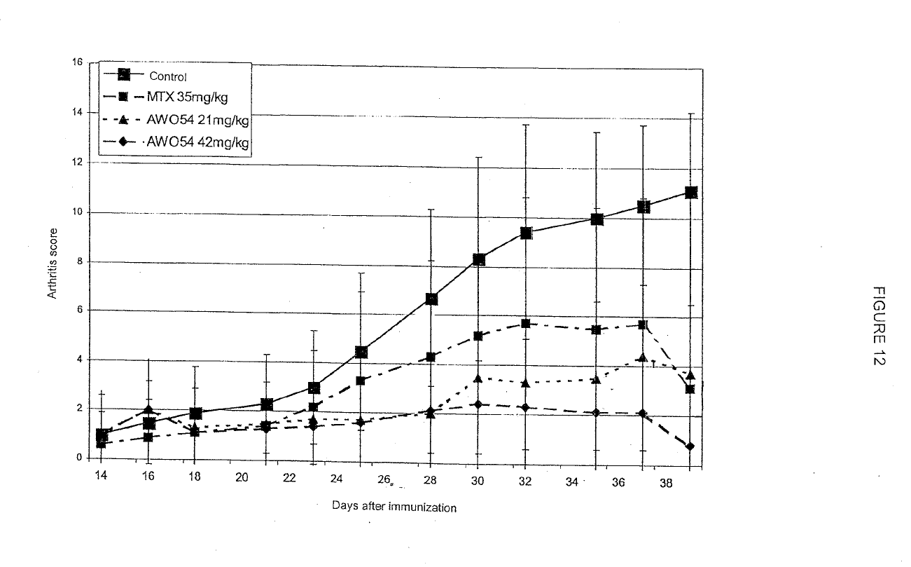

FIG. 10: a graphical illustration showing the course of RA score in a collagen-induced arthritis model.

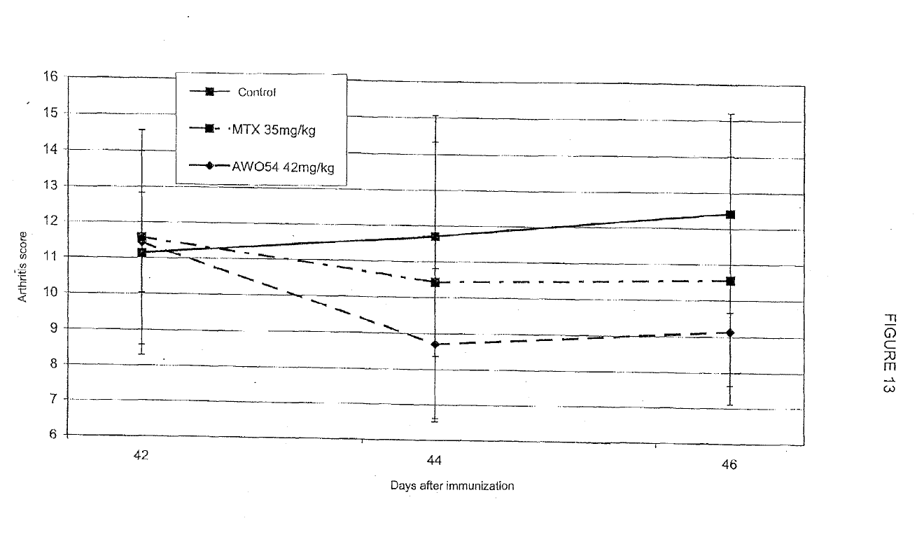

FIG. 11: a graphical illustration showing the course of arthritis occurrence in a collagen-induced arthritis model with an early treatment protocol (beginning of treatment as of day 14 of immunization).

FIG. 12: a graphical illustration showing the course of arthritis score in a collagen-induced arthritis model with an early treatment protocol (beginning of treatment as of day 14 of immunization).

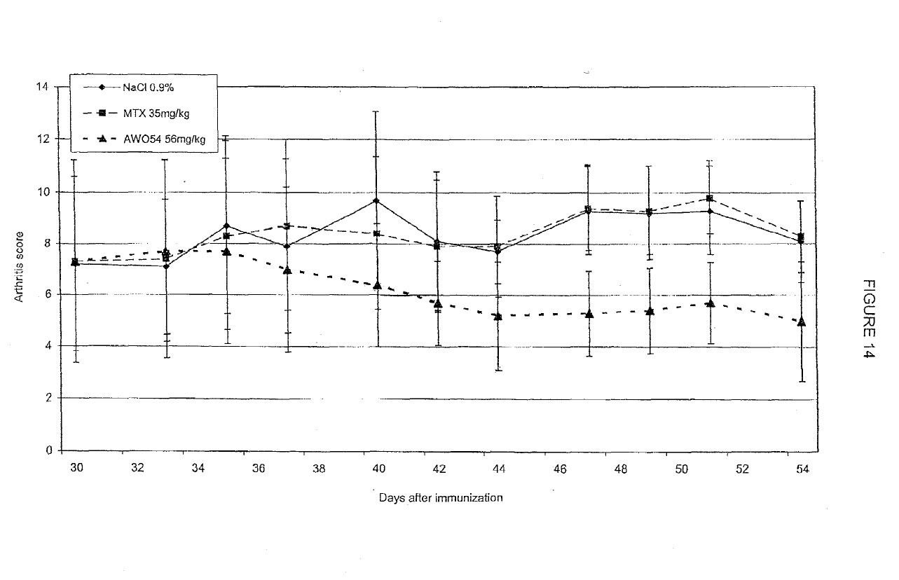

FIG. 13: a graphical illustration showing the course of arthritis score in a collagen-induced arthritis model with a late treatment protocol (beginning of treatment as of day 42 of immunization).

FIG. 14: a graphical illustration showing the course of arthritis score in a collagen-induced arthritis model with an intermediate treatment protocol (beginning of treatment as of day 30 of immunization).

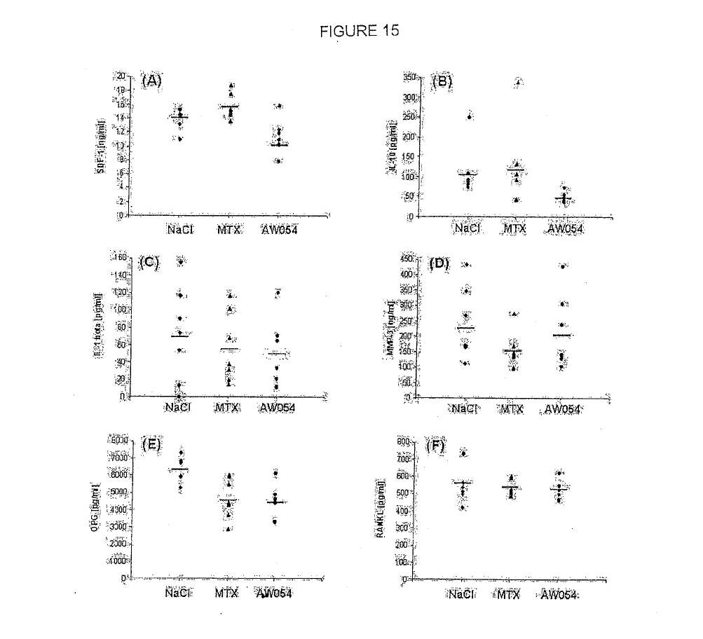

FIG. 15: results of the measurement of cytokine, chemokine and enzyme concentrations in a collagen-induced arthritis model.

The following examples explain the present invention in more detail without being limited thereto.

### EXAMPLES

### Example 1

### Preparation of EMC-D-Ala-Phe-Lys-Lys(γ-MTX)-OH

DIEA (27.2 μL, 159 μmol) and HATU (13.29 mg, 34.96 μmol) are successively added to a solution of methotrexate-α-tert.-butyl ester (MTX-α-OtBu) (17.85 mg, 34.96 μmol) in 150 μL of anhydrous DMF. After 2 min of treatment in an ultrasonic bath, the reaction mixture is added to a solution of EMC-D-Ala-Phe-Lys(Boc)-Lys-OH (31.78 μmol) in 1.5 mL of anhydrous DMF and stirred for 1 hour at room temperature. Subsequently, the reaction mixture is added to 100 mL of diethyl ether, the precipitate is centrifuged off, washed twice with diethyl ether and dried in vacuum. To cleave the protective groups, the raw product is treated for 1 hour with 5 mL of dichloromethane/TFA 1:1 and added to 100 mL of diethyl ether, the precipitate is centrifuged off, washed twice with diethyl ether and dried in vacuum. After preparative HPLC (C18 reverse phase, MeCN/water 30:70, 0.1% TFA) and lyophilization, EMC-D-Ala-Phe-Lys-Lys(γ-MTX)-OH is obtained as a light yellow solid substance.

ESI-MS (4.0 kV, MeCN): m/z (%) 1122.3 ([M+H]⁺, 100), 1144.4 ([M+Na]⁺, 73)

### Example 2

### Bonding of EMC-D-Ala-Phe-Lys-Lys(γ-MTX)-OH to HSA in Human Plasma

A sample of human blood plasma is incubated with EMC-D-Ala-Phe-Lys-Lys(γ-MTX)-OH (200 μM) for 2 min at 37° C. and subsequently analyzed by means of chromatography on a C₁₈-RP-HPLC column (Symmetry® 300-5 4.6×250 mm by Waters with pre-column filter) by gradient elution (flow: 1.2 mL/min; eluent A: 30% 20 mM K₂HPO₄ pH 7, 70% acetonitrile; eluent B: 85% 20 mM K₂HPO₄ pH 7, 15% acetonitrile; gradient: 20 min eluent β isocratic, 25 min 0-100% eluent A linear, 5 min eluent A isocratic). A detection at a wavelength of 300 nm characteristic for MTX derivatives shows an almost complete decrease of the prodrug peak and an increase in absorption at a retention time of albumin (t˜32 min) (see FIG. 1).

Moreover, a further analysis after 24 hours reveals, on the basis of the corresponding peak areas, that the loss of MTX is less than 10%.

### Example 3

### Bonding of EMC-Arg-Ala-Phe-Met-Lys(γ-MTX)-OH (C162) to HSA in Human Plasma

A sample of human blood plasma is incubated with EMC-Arg-Ala-Phe-Met-Lys(γ-MTX)-OH (200 μM) for 2 min at 37° C. and subsequently analyzed by means of chromatography on a C₁₈-RP-HPLC column (Symmetry® 300-5 4.6×250 mm by Waters with pre-column filter) by gradient elution (flow: 1.2 mL/min; eluent A: 30% 20 mM K₂HPO₄ pH 7, 70% acetonitrile; eluent B: 85% 20 mM K₂HPO₄ pH 7, 15% acetonitrile; gradient: 20 min eluent β isocratic, 25 min 0-100% eluent A linear, 5 min eluent A isocratic). A detection at a wavelength of 370 nm characteristic for MTX derivatives shows an almost complete decrease of the prodrug peak and an increase in absorption at a retention time of albumin (t=40 min) (see FIG. 2).

A repetition of the test with human blood plasma having been incubated with EMC (1000 μM) for 5 min in advance, which results is a blocking of the cysteine-34-group of albumin, does not show a bonding of the prodrug to albumin during a subsequent incubation with EMC-Arg-Ala-Phe-Met-Lys(γ-MTX)-OH. In the chromatogram, merely the free prodrug can be detected at 370 nm.

### Example 4

### Enzymatic Cleavage of the Albumin Conjugate from EMC-D-Ala-Phe-Lys-Lys(γ-MTX)-OH by Cathepsin B and Plasmin

Preparation of the albumin conjugate: 4.00 mg of EMC-D-Ala-Phe-Lys-Lys(γ-MTX)-OH are dissolved in 8 mL of a 5% HSA solution (Octopharm) at room temperature and shaken at 37° C. for 2 hours. Subsequently, the sample is brought to a concentration of 700 μM by concentration with Centriprep® disposable concentrators.

Cleavage by plasmin: Now, 100 μL of the solution of the albumin conjugate are diluted with 500 μL buffer (4 mM sodium phosphate, 150 mM NaCl, pH 7.4), 20 μL of human plasma plasmin (370 mU) are added and the mixture is incubated at 37° C. The determination of the cleavage products is performed with the HPLC method described in Example 2 (FIGS. 3 and 4).

Cleavage by cathepsin B: Now, 180 μL of the solution of the albumin conjugate are diluted with 270 μL buffer (50 mM sodium acetate, 100 mM NaCl, 4 mM EDTA*2 Na, 8 mM L-cysteine, pH 5.0), 90 μL of human cathepsin B (2.1 U) are added and incubated at 37° C. The determination of the cleavage products is performed with the HPLC method described in Example 2 (FIGS. 5 and 6).

Result: After one and four hours, respectively, of incubation with the enzymes, the formation of H-Lys(γ-MTX)-OH as a cleavage product at ˜4 min can be observed in both cases. In addition, it is evident that in the course of time, the concentration of the albumin conjugate decreases and the concentration of the cleavage product increases. The cleavage product thus results from the proteolytic cleavage of the Lys-Lys bond.

### Example 5

### Cleavage of HSA-EMC-D-Ala-Phe-Lys-Lys(γ-MTX)-OH in the Homogenate of a Human Ovarian Xenograft (OVCAR-3)

Preparation of the Tumor Homogenate: The Tumor Material is Comminuted by Means of a scalpel, and 200 mg of the mass are homogenized in a shaker with 800 μL buffer (Tris-buffer pH 7.4) with addition of 3-4 glass beads. Subsequently, centrifugation is carried out at 4° C. and the supernatant is aliquoted to 200 μL.

Now, 100 μL of the solution of the albumin conjugate EMC-D-Ala-Phe-Lys-Lys(γ-MTX)-OH described in Example 4 are diluted with 500 μL of a homogenate solution (homogenate, 1:2 diluted with buffer [4 mM sodium phosphate, 150 mM NaCl, pH 7.4]) and incubated at 37° C. The determination of the cleavage products is performed with the HPLC method described in Example 2 (FIG. 7).

Result: After four hours of incubation with OVCAR-3 tumor homogenate, the formation of H-Lys(γ-MTX)-OH as a cleavage product can be observed.

### Example 6

### Cleavage of EMC-Arg-Ala-Phe-Met-Lys(γ-MTX)-OH (C162) in Synovial Fluids of Patients Suffering from RA

Preparation of the albumin conjugate: 4.00 mg of EMC-Arg-Ala-Phe-Met-Lys(γ-MTX)-OH are dissolved in 8 mL of a 5% HSA solution (Octopharm) at room temperature and shaken at 37° C. for 2 hours. Subsequently, the sample is brought to a concentration of 700 μM by concentration with Centriprep® disposable concentrators.

Now, 70 μL of the solution of the albumin conjugate of EMC-Arg-Ala-Phe-Met-Lys(γ-MTX)-OH are diluted with 140 μL synovial fluid (synovial fluid of six patients suffering from rheumatoid arthritis, diluted 1:1 with distilled water) and incubated at 37° C. The determination of the cleavage products is performed with the HPLC method described in Example 2 (FIG. 8).

Result: After four hours of incubation with the synovial fluid of patients suffering from rheumatoid arthritis, the formation of H-Lys(γ-MTX)-OH as a cleavage product can be observed.

### Example 7

### Effectiveness of EMC-D-Ala-Phe-Lys-Lys(γ-MTX)-OH and EMC-Arg-Arg-Ala-Met-Lys(γ-MTX)-OH In Vivo (Tumor-Inhibiting Properties)

The biological data listed below and in FIG. 9 show an increased in-vivo effectiveness of EMC-D-Ala-Phe-Lys-Lys(γ-MTX)-OH (AW054-EMC) and EMC-Arg-Arg-Ala-Met-Lys(γ-MTX)-OH(C175) compared to free methotrexate.

Animals: nude mice NMRI; tumor model: OVCAR-3 (ovarian carcinoma growing subcutaneously)
Therapy: day 7, 14, 21, 28; i. v. (10 mM sodium phosphate/5% D-glucose buffer pH 6.4); dosages relate to methotrexate equivalents.

| | dosage | change of body | T/C [%] |
|---|---|---|---|
| substance | [mg/Kg] | weight [%] | maximum |
| MTX | 4 × 100 | +19 | 69 |
| AW054-EMC | 4 × 15 | +12 | 29 |
| C175 | 3 × 15 | +7 | 40 |

### Example 8

### Effectiveness of EMC-D-Ala-Phe-Lys-Lys(γ-MTX)-OH In Vivo (Antirheumatic Properties)

The biological data listed below and in FIG. 10 show an increased in-vivo effectiveness of EMC-D-Ala-Phe-Lys-Lys(γ-MTX)-OH (AW054-EMC) compared to free methotrexate.

Animals: mice (m, DBA/1; model: collagen-induced arthritis model)
Therapy: day 30, 34, 37, 41, 44, 48; i. v. (10 mM sodium phosphate/5% D-glucose buffer pH 6.4); dosages relate to methotrexate equivalents.

| | dosage | change of body | RA score |
|---|---|---|---|
| substance | [mg/Kg] | weight [%] | Tag 55 |
| control | — | +9.5 | 8.10 |
| MTX | 6 × 35 | +6.9 | 8.30 |
| AW054-EMC | 6 × 20 | −0.2 | 5.00 |

The biological data listed below, in FIGS. 11 to 15 and Table 1 again show an increased in-vivo effectiveness of EMC-D-Ala-Phe-Lys-Lys(γ-MTX)-OH (AW054-EMC) compared to free methotrexate.

Animals: mice (m, DBA/1; model: collagen-induced arthritis model)

Therapy: twice a week as of day 14, 42 and 30, respectively; i. v. (10 mM sodium phosphate/5% D-glucose buffer pH 6.4); dosages relate to methotrexate equivalents. Substances and dosages are indicated in FIGS. 11 to 15.

The measurements of protein concentration in serum after a 6-day treatment are performed by means of ELISA (commercially available from R&D Systems Wiesbaden Germany) according to the protocol of the manufacturer.

The following Table 1 shows the reaction to the treatment with MTX or different dosages of AW054 compared to the NaCl control in the early treatment protocol. The treatment with AW054 leads to a reduced occurrence of developed arthritis at the end of the test, reduces the mean arthritis score, prolongs the time until the first occurrence of arthritis and induces an improvement or even an abatement of developed arthritis.

TABLE 1

| | (±standard deviation) | | | | | | | |
|---|---|---|---|---|---|---|---|---|
| | NaCl | | MTX | | AW054 | | AW054 | |
| | control | | 35 mg/kg | | 21 mg/kg | | 42 mg/kg | |
| | n = 29 | | n = 15 | | n = 14 | | n = 11 | |
| occurrence of arthritis at the | 29 | (100) | 9 | (60) | 9 | (64) | 2 | (18) |
| end of the test (%) | | | | | | | | |
| mean arthritis score at the | 11.1 | (±3.1) | 3.1 | (±3.4) | 3.7 | (±4.3) | 0.8 | (±2.4) |
| end of the test | | | | | | | | |
| mean period of time until | 8.3 | (±5.7) | 17.3 | (±10.8) | 6.8 | (±7.6) | 11.5 | (±11.1) |
| breakout of renewed | | | | | | | | |
| arthritis after beginning of | | | | | | | | |
| treatment in days | | | | | | | | |
| improved or achieved | 0 | (0) | 8 | (53) | 10 | (71) | 8 | (73) |
| abatement after created | | | | | | | | |
| disease | | | | | | | | |

It becomes evident from the examples that after incubation with human blood plasma, the corresponding albumin conjugate is substantially formed already after 2 min. The conjugates exhibit sufficient plasma stability, and an effective cleavage in the presence of both human plasmin and cathepsin B can be observed. The cleavage results in the formation of e.g. H-Lys(γ-MTX)-OH, which constitutes the only low-molecular cleavage product. Then, however, this cleavage product is not cleaved into MTX and lysine any more, and the tests in vivo correspondingly suggest that the MTX-lysine derivative of the present invention is per se highly active. In comparison to methotrexate, it exhibits increased efficiency with a much lower dosage. In the case of the collagen-induced arthritis model, it is about 20% of the corresponding methotrexate equivalent dosage. Moreover, the derivative is active for a longer period of time, and the serum concentrations of e.g. SDF-1, OPG and IL-10 are significantly reduced.

1. A methotrexate derivative of the structural formula I: 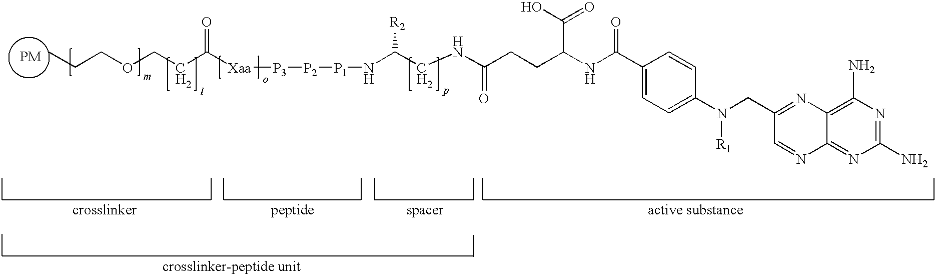
wherein
R₁=H or CH₃
R₂=H or COOH
P₁=lysine, methionine, alanine, proline or glycine
P₂=leucine, phenylalanine, methionine, alanine, proline or tyrosine
P₃=D-alanine, alanine, D-valine, valine, leucine or phenylalanine
Xₐₐ=amino acid with alkaline side chain
m=0 to 6
n=0 to 5
o=0 to 2
p=1 to 10
PM is a protein-binding group.

2. The methotrexate derivative according to claim 1, wherein PM is selected from a group consisting of a maleinimide group, a 2-dithiopyridyl group, a halogen acetamide group, a halogen acetate group, a disulphide group, an acrylic acid ester group, a monoalkyl maleic acid ester group, a monoalkyl maleamine acid amide group, an N-hydroxy succinimidyl ester group, an isothiocyanate group and an aziridine group, which may be optionally substituted.

3. The methotrexate derivative according to claim 2, wherein PM is a maleinimide group, which may be optionally substituted.

4. The methotrexate derivative according to claim 3, wherein m=0 and n=4.

5. The methotrexate derivative according to claim 3, wherein m=3 and n=1.

6. The methotrexate derivative according to claim 1, wherein R₁=CH₃.

7. The methotrexate derivative according to claim 1, wherein R₂=COOH and p=4.

8. The methotrexate derivative according to claim 1, wherein P₁=lysine, alanine or methionine.

9. The methotrexate derivative according to claim 1, wherein P₂=phenylalanine, methionine, alanine or tyrosine.

10. The methotrexate derivative according to claim 1, wherein P₃=D-alanine, alanine, D-valine, valine or phenylalanine.

11. The methotrexate derivative according to claim 8, wherein P₁=lysine, P₂=leucine or phenylalanine and P₃=alanine, D-alanine, valine or D-valine.

12. The methotrexate derivative according to claim 11, wherein P₂=leucine and P₃=D-valine.

13. The methotrexate derivative according to claim 11, wherein P₂=leucine and P₃=valine.

14. The methotrexate derivative according to claim 11, wherein P₂=phenylalanine and P₃=D-alanine.

15. The methotrexate derivative according to claim 11, wherein P₂=phenylalanine and P₃=alanine.

16. The methotrexate derivative according to claim 8, wherein P₁=methionine, P₂=methionine, alanine or phenylalanine and P₃=alanine or phenylalanine.

17. The methotrexate derivative according to claim 16, wherein P₂=alanine and P₃=phenylalanine.

18. The methotrexate derivative according to claim 16, wherein P₂=phenylalanine and P₃=alanine sind.

19. The methotrexate derivative according to claim 16, wherein P₂=methionine and P₃=alanine.

20. The methotrexate derivative according to claim 16, wherein P₂=methionine and P₃=phenylalanine.

21. The methotrexate derivative according to claim 1, wherein o=0.

22. The methotrexate derivative according to claim 1, wherein Xₐₐ=arginine, lysine or histidine.

23. The methotrexate derivative according to claim 22, wherein Xₐₐ=arginine and o=2.

24. A method for producing methotrexate derivatives according to claim 1, comprising reacting a methotrexate derivative having the general structural formula II 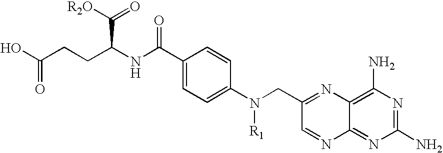
wherein
R₁=CH₃, H or COCF₃
R₂=C(CH₃)₃, an alkoxy-substituted benzyl group or a trialkyl silyl group, in the presence of a carboxylic acid activation reagent with addition of catalysts/auxiliary bases with a crosslinker-peptide unit of the general structural formula III 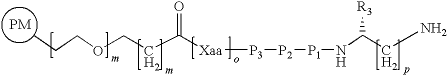
wherein
R₃=H, COOH or COOtBu
P₁=lysine, methionine, alanine, proline or glycine
P₂=leucine, phenylalanine, methionine, alanine, proline or tyrosine
P₃=D-alanine, alanine, D-valine, valine, leucine or phenylalanine
Xₐₐ=amino acid with alkaline side chain
m=0 to 6
n=0 to 5
o=0 to 2
p=1 to 10
PM is a protein-binding group,
wherein possible nucleophilic groups are present, optionally protected by protective groups, at P₁, P₂ and Xaa and treated with an acid, optionally with addition of cation-scavenging reagents, in a second step.

25. The method according to claim 24, wherein the carboxylic acid activation reagent is selected from the group consisting of N,N′-diisopropyl carbodiimide, N,N′-dicyclohexyl carbodiimide, (benzotriazol-1-yloxy)tris(dimethylamino)phosphonium hexafluorophosphate, 2-chloro-1-methylpyridinium iodide and O-(azabenzotriazol-1-yl)-N,N,N′,N′-tetramethyluronium hexafluorophosphate.

26. The method according to claim 24, wherein the catalyst/auxiliary base is selected from the group consisting of trialkylamines, pyridine, 4-dimethylaminopyridine (DMAP) and hydroxybenzotriazole (HOBt), or a combination thereof.

27. The method according to claim 24, wherein O-(azabenzotriazol-1-yl)-N,N,N′,N′-tetramethyluronium hexafluorophosphate in connection with N-ethyldiisopropylamine is used as a carboxylic acid activation reagent

28. The method according to claim 24, wherein hydrogen chloride is used as an acid in the second step.

29. The method according to claim 24, wherein trifluoroacetic acid is used as an acid in the second step.

30. The method according to claim 24, wherein in the second step, the cation-scavenging reagent is selected from the group consisting of water, phenol, thioanisole, diisopropylsilane and 1,2-ethane dithiole, or a combination thereof.

31. The method according to claim 24, wherein methotrexate-α-tert.-butylester is reacted with ((((6-maleinimidohexanoyl)D-alanyl)phenylalanyl)tert.-butoxylcarbonyllysyl)lysine-trifluoracetate using O-(azabenzotriazol-1-yl)-N,N,N′,N′-tetramethyluronium hexafluorophosphate in connection N-ethyldiisopropylamine and treated with trifluoroacetic acid in a second step.

32. A medicament comprising a methotrexate derivative according to claim 1, together with one or more pharmaceutically acceptable auxiliary agents.

33. A method of treating cancer comprising administering a methotrexate derivative according to claim 1 to a mammal.

34. A method of treating rheumatic disease comprising administering a methotrexate derivative according to claim 1 to a mammal.
